(19) United States
(12) Patent Application Publication
(10) Pub. No.: US 2022/0379021 A1
(43) Pub. Date: Dec. 1, 2022

(54) MULTIPURPOSE INFUSION MONITORING AND CONTROLLING UNIT AND METHOD THEREOF

(71) Applicant: Vijay Mohantal Mehta, Pune (IN)

(72) Inventor: Vijay Mohanlal Mehta, Pune (IN)

(21) Appl. No.: 17/770,432

(22) PCT Filed: Jul. 13, 2020
(86) PCT No.: PCT/IN2020/050603

§ 371 Date: Apr. 20, 2022

(30) Foreign Application Priority Data
Oct. 22, 2019 (IN) 201921042897

(51) Int. Cl. A61M 5/168; A61M 5/14

(52) U.S. Cl. CPC A61M 5/1689; A61M 5/1411; A61M 5/16877; A61M 5/16813; A61M 2205/33

(57) ABSTRACT

Disclosed herein a real time multipurpose infusion monitoring and controlling unit comprising power source, monitor, micro controller and drop rate sensors, wherein said unit is performing employing horizontal screw mechanism adapted to monitor and control the drip by locking the airdrop in the saline tube to make the drip mechanism more accurately and efficiently.

### FIELD OF THE INVENTION

This invention, in general relates to an Infusion monitoring and controlling unit and method thereof. More particularly, the present invention provides a real time multipurpose infusion monitoring and controlling unit.

### BACKGROUND OF THE INVENTION

Infusion is most needed unit to deliver critical fluids, for every day patient care in big and small hospitals, primary health centers, day care centers, ambulances etc. However, delivery of the fluids in controlled and accurate manner is extremely important to avoid certain side effect and health hazardous for patients.

Various approaches have been disclosed in the prior arts in relation to develop infusion device and system to drip infusions.

CN103785080 (A) titled Method for implementing multifunctional infusion monitor and software related to multifunctional infusion monitor discloses A method for implementing a multifunctional infusion monitor and software related to the multifunctional infusion monitor includes the steps of combining various types of hardware on the multifunctional infusion monitor with the software, inputting, checking and printing infusion information, simulating the infusion process in real time, dynamically demonstrating the infusion process, accurately adjusting and setting the infusion speed, conducting online alarm setting and the like on various infusion parameters, and allowing operators/patients to use, check and manage various types of other information and functions through the functions of the software itself or through matching of the functions of the software and other hardware.

CN103948986 (A) titled Real-time infusion monitoring system for wisdom medical treatment discloses the invention discloses a real-time infusion monitoring system for wisdom medical treatment, and relates to a sensor technology, an embedded technology, a wireless fidelity (WIFI) technology and the like. The real-time infusion monitoring system comprises an infusion monitor, a monitoring server and a mobile terminal which have information transmission with one another by WIFI, wherein the infusion monitor comprises a center controller, a monitor and a speed controller. The real-time infusion monitoring system is characterized in that the infusion monitor is used for sampling and calculating infusion dripping speed and liquid level, the infusion speed is adjusted by the speed controller, and the results are transmitted into the monitoring server in real time; the monitoring server of a nurse station is supported by an information base, and is used for providing the infusion dosage and the recommended speed value according to the state of an illness and the pharmacology, in comparison with the real-time speed information of the monitor, realizing the remote quantitative regulation and control for the ward infusion process by a WIFI network; the mobile terminal is used for receiving the emergency call transmitted by a ward monitor, sending out a remote processing instruction for remote infusion regulation and control, and stopping infusion in a remote way when necessary. The system is high in degree of automation, accurate, real-time, intelligent and practical.

CN104784771 titled Intelligent infusion device discloses The invention discloses an intelligent infusion device. The intelligent infusion device comprises an infusion bottle control mechanism, a puncture outfit mechanical arm mechanism, an infusion liquid dynamic monitoring mechanism, an infusion liquid temperature control mechanism, an automatic needle plugging and unplugging mechanism, a central control station and a central control unit. The central control unit corresponds to the puncture outfit mechanical arm mechanism, the liquid dynamic monitoring mechanism, the liquid temperature control mechanism and the automatic needle plugging and unplugging mechanism. The intelligent infusion device has the advantage that automatic integration in the infusion process can be achieved, which is concretely embodied in that an infusion bottle can be automatically changed; infusion dripping speed, flowing speed and liquid bubbles are monitored, the infusion dripping speed and flowing speed are controlled, and the bubbles are eliminated in time; in the infusion process, an infusion tube is heated, and heating temperature is automatically controlled; through control of mechanical arms, automatic needle plugging and unplugging are achieved; the central receiver controls controllers of all units, so that control over all stages in the infusion process is achieved.

CN203736619 (U) titled Drip infusion monitoring device discloses a utility model discloses a drip infusion monitoring device. The drip infusion monitoring device comprises a power supply, a microprocessor, a dripping speed detector, a liquid level detector, a dripping speed regulator, a keyboard display and an alarm, wherein the power supply supplies power for the microprocessor, the dripping speed detector, the liquid level detector, the dripping speed regulator, the keyboard display and the alarm; the microprocessor are respectively and electrically connected with the dripping speed detector, the liquid level detector, the dripping speed regulator and the keyboard display; the microprocessor is in wireless communication connection with the alarm; meanwhile, the dripping speed regulator adopts a motor. The drip infusion monitoring device solves the problems that in the prior art, medical negligence probability is large due to manual infusion speed control and manual residual liquid monitoring, and no effective device for special patients can be used for controlling infusion speed and monitoring residual liquid. The drip infusion monitoring device can intelligently, remotely and concentratedly control infusion speed and monitor residual liquid; therefore, the comprehensive management capability of hospital is improved, and medical negligence probability is reduced.

However, currently available solutions as disclosed above have their own restrictions in developing different types of system and method for infusion unit and mechanism such as, cost effectiveness, monitor and control automatically while performing the operation, accuracy and precision in the performance, effectively using at various places including remote places etc.

It would be desirable, therefore, there is need to develop an improved infusion unit to perform the infusion of drip infusions effectively with cost efficient manner.

### SUMMARY OF THE INVENTION

The various objectives and embodiments of the present invention as presented herein are understood to be illustrative of the present invention and not restrictive thereof and are non-limiting with respect to the scope of the invention.

It is one objective of the present invention to provide a cost effective infusion monitoring and controlling unit, wherein said unit is monitoring and controlling the gravity-fed drip infusions.

It is another objective of the present invention to provide a compact and portable infusion unit capable of use anywhere.

It is another objective of the present invention to provide an infusion unit which is reliable and accurate.

It is another objective of the present invention to provide an infusion unit adapted to work on Battery, main, or solar or other renewal energy power.

It is another objective of the present invention to provide an infusion unit, wherein said unit is adapted to employ with other ad-on healthcare equipment.

It is another objective of the present invention to provide an infusion unit, wherein said unit is adapted to control the infusion volume of different drugs.

It is another objective of the present invention to provide an infusion unit, wherein said unit is adapted to keep record of both quantitative and qualitative list of drugs to be infused.

It is another objective of the present invention to provide an infusion unit, wherein said unit is adapted to monitor temperature to get accurate digital temperature.

It is another objective of the present invention to provide an infusion unit, wherein said unit is a complete web based unit.

It is another objective of the present invention to provide an infusion unit, wherein said unit is capable of setting multiple reminders for patient health management thus making the nursing job easier.

The above and other objectives of the present invention are achieved according to the following embodiments of the present invention. However the disclosed embodiments are exemplary based on the preferred and best mode of the invention and not limit the scope of the present invention.

In accordance with one embodiment of the present invention, there is provided a real time multipurpose infusion monitoring and controlling unit comprising power source, monitor, micro controller and drop rate sensors, wherein said unit is performing employed horizontal screw mechanism adapted to monitor and control the drip by air locking the saline tube to make the drip mechanism more accurate and efficient.

In accordance with one embodiment of the present invention, there is provided a real time multipurpose infusion monitoring and controlling unit, wherein the screw type mechanism employed for pushing and controlling the airdrop in a saline tube, wherein said mechanism is performed by an assembly comprising a compact DC motor, a gear box, a shaft and, a plunger on the shaft, wherein once we supply the power to the motor, the movement of motor starts which is adapted to provide multi-steps to get the desired drop rates and also provide torque of around 2 kg to create air lock of the saline tube to prevent air embolism in patient.

In accordance with one embodiment of the present invention, there is provided a real time multipurpose infusion monitoring and controlling unit, wherein the screw type mechanism is having three positions as an air lock position, required drop rate position and zero position.

In accordance with just above embodiment of the present invention, wherein said air lock position is fully lock position to adjust and control the droplets by obliterating the tube, required drop rate position is the position at which the drug is infused in patient at the required drop rate and zero position is the position when the tube is unlocked once the infusion is over.

In accordance with one embodiment of the present invention, there is provided a real time multipurpose infusion monitoring and controlling unit, wherein said infusion unit is monitoring and controlling the gravity-fed drip infusions.

In accordance with one embodiment of the present invention, there is provided a real time multipurpose infusion monitoring and controlling unit, wherein said infusion unit is compact and portable and is used anywhere in ICU as well as in general ward and wherein safely monitors the intravenous delivery of all kinds of fluids including chemotherapy treatment.

In accordance with one embodiment of the present invention, there is provided a real time multipurpose infusion monitoring and controlling unit comprising power source, monitor, micro controller and drop rate sensors, wherein said sensors are auto locking type universal drop rate sensor which operates with all kinds of giving sets without the use of special adaptors or other extra hardware.

In accordance with one embodiment of the present invention, there is provided a real time multipurpose infusion monitoring and controlling unit, wherein said unit is used with multiple ad-on equipments including B P machine, temperature sensing machine, ECG, Pulse check tool and the like.

In accordance with one embodiment of the present invention, there is provided a real time multipurpose infusion monitoring and controlling unit, wherein said unit has in-built embedded high end micro controller which is capable of communicating unit to unit by RF data stream and which finally does data logging on a cloud or web server where it can viewed or accessed from anywhere in the world.

In accordance with one embodiment of the present invention, there is provided a real time multipurpose infusion monitoring and controlling unit, wherein said unit is having universal mounting means that equipment can be standardized across the hospital or institution with the unit easily switched from one treatment to another.

These and other embodiments, processes and features of the invention will become more fully apparent when the following detailed description is read with the accompanying drawing figures. However, both the foregoing summary of the invention and the following detailed description of it represent one potential embodiment, and are not restrictive of the invention or other alternate embodiments of the invention.

### BRIEF DESCRIPTION OF DRAWINGS

To illustrate the solutions according to the embodiments of the present disclosure more clearly, the accompanying drawings needed for describing the embodiments are introduced below briefly. Apparently, the accompanying drawings in the following descriptions merely show some of the embodiments of the present disclosure, and persons skilled in the art may obtain other drawings according to the accompanying drawings without creative efforts.

FIG. 1 illustrates a schematic diagram of link among the multipurpose infusion drip apparatus, cloud server and computer system.

FIG. 2 shows the internal components of said apparatus along with the mounting clamp.

FIG. 3 shows the back portion of said apparatus along with mounting clamping on stand.

FIGS. 4, 5 and 6 shows the different perspective views of horizontal screw tool.

### DETAILED DESCRIPTION OF THE INVENTION

The solutions of the present disclosure are to be clearly described in the following with reference to the accompanying drawings. It is obvious that the embodiments to be described are only a part rather than all the embodiments of the present disclosure. All other embodiments obtained by persons skilled in the art based on the embodiments of the present disclosure without creative efforts shall fall within the protection scope of the present disclosure.

According to one non-limiting embodiment of the present invention, a real time multipurpose infusion monitoring and controlling unit comprising power source, monitor, micro controller and drop rate sensors, wherein said unit is performing employing horizontal screw mechanism adapted to monitor and control the drip by air-locking the saline tube to make the drip mechanism more accurately and efficiently and wherein the screw type mechanism employed for pushing and controlling the airdrop in a saline tube, wherein said mechanism is performed by an assembly comprising a compact DC motor, a gear box, a shaft and, a plunger on the shaft, wherein once we supply the power to the motor, the movement of motor starts which is adapted to provide multi-steps to get the desired drop rates and also provide torque of around 2 kg to create air lock of the saline tube to prevent air embolism in patient enabling air locking the saline tube to make the drip mechanism more accurate and efficient.

In accordance with one embodiment of the present invention, there is provided a real time multipurpose infusion monitoring and controlling unit, wherein the screw type mechanism is having three positions as an air lock position, required drop rate position and zero position wherein said air lock position is fully lock position to adjust and control the droplets by obliterating the tube, required drop rate position is the position at which the drug is infused in patient at the required drop rate and zero position is the position when the tube is unlocked once the infusion is over.

Advantage of the present invention is to obviate the drawback associated with the existing arts and provide lightweight, portable and multifunctional infusion adapted to use anywhere including ICU and general wards of any health institutions.

According to the present invention, there is provided a microcontroller based infusion unit enabling the user to change the drop rate online with reference to infusion time, wherein customized settings on real time basis is provided and displaying all concerned information and indication along with audio visual alarm for set parameters.

According to the present invention, there is provided a microcontroller based infusion unit using the concept of fibre optics and electronics in combination to monitor the each individual drop of fluid leaving the given set of intravenous drip accurately and constantly recalculates and displays the flow rate in drops per minute digitally and can control drop rate online with reference to infusion time thus delivering accurate measurement of drugs and liquids safely as prescribed by staff and reduces workload on nurse staff.

In general the flow of gravity-fed drips can be affected by a number of factors, it is imperative that they are checked regularly. This takes nursing staff away from their other important tasks and interrupts the efficient running of the ward. With the improved infusion unit tremendously reduces workload on our nursing staff so they can give more time directly to patient care. It digitally monitors drops for any stoppage, any high and low deviation, completion of total or pre set volume etc. and makes nurses, patients and their family members 100% free from anxiety & continuously watching infusion for any problems.

The infusion monitoring and controlling unit according to present invention is a precision instrument which monitors gravity-fed drip infusions. It makes a significant contribution to the safety of patients who are receiving treatment by intravenous drip.

Various additional facility is provided in the current infusion unit such as selection of adult or micro infusion set (pediatric) before you start infusion, registers every drop of fluid and constantly recalculates and displays the flow rate in drops per minute, if the flow rate deviates from a specified range, ward staff are alerted quickly with both audible and visual alarms. During the night, a bag light alarm can be used to silently alert staff, infusion monitor allows staff to see at a glance how much fluid has been infused, accurate measurements of drugs and liquids can be delivered safely as prescribed with staff alerted once the specified quantity has been infused, customized settings for starting infusion with pre-defined total and preset volume so no need of setting it every time, use scroll on-off facility to monitor any parameter continuously, auto continuation facility to infuse balance volume once pre set volume is over by pressing single button, online change drop rate facility with reference to infusion time, online indication and display for battery charge percentage so you will never be without power during infusion, also alerts for low battery, Auto-Off and On facility is provided in the unit therefore there is no need to go to each and every unit in order to shut them down if it is being setup for an automatic-Off and On facility, said unit is capable of controlling the infusion volume of different drugs, said unit is capable of keeping record of both quantitative and qualitative list of drugs to be infused in patients. The said unit is also capable of monitoring temperature to get accurate digital temperature, wherein said accurate digital temperature helps in continuous monitoring as it is a very critical factor and might affect the infusion in patient further leading to negative consequences. The infusion unit is a complete web based unit which is also capable of setting multiple reminders for patient health management thus making the nursing job easier.

According to the present invention, said infusion unit operates through a two way communication method that further comprises of communicating through a mesh network for unit to unit communication, receiving information on web gateway with the help of Wi-Fi or GSM and transmitting the same to web server and conveying the integrated information about the condition of patient on web application of suitable user friendly electronic device.

According to the present invention, the infusion monitor provided herein monitors the flow rate of intravenous drips, but it does not come into contact with the fluid hence the risks of contamination or extravasations are minimized.

The infusion unit disclosed herein can run on inbuilt rechargeable battery for 24 hrs, or mains supply 230 VAC, 50 Hz, or 1 w 12 volt solar panel, or any 12 volt external battery

According to the present invention, all data is logged when hold facility used meaning by infusion monitor keeps accurate records of all infusion activity when infusion is put on hold for any reason like x-ray etc and restarts from same point again even if unit is switched off.

Nurse's time is freed up dramatically when using said infusion unit as disclosed in the present invention, as the device constantly monitors drip rates allowing staff to concentrate on other vital tasks. If there is a problem, staffs are alerted. Otherwise, they can get on with their jobs.

The infusion unit has universal mounting means that equipment can be standardized across the hospital or institution with the unit easily switched from one treatment to another. It can also be wall mounted if required. It is very easy to set and use equipment for medical/paramedical staff and nursing staff in particular.

An interesting model of using said infusion unit is provided according to the present invention, wherein user can use prepaid facility to use said unit which effectively reduces the cost of use.

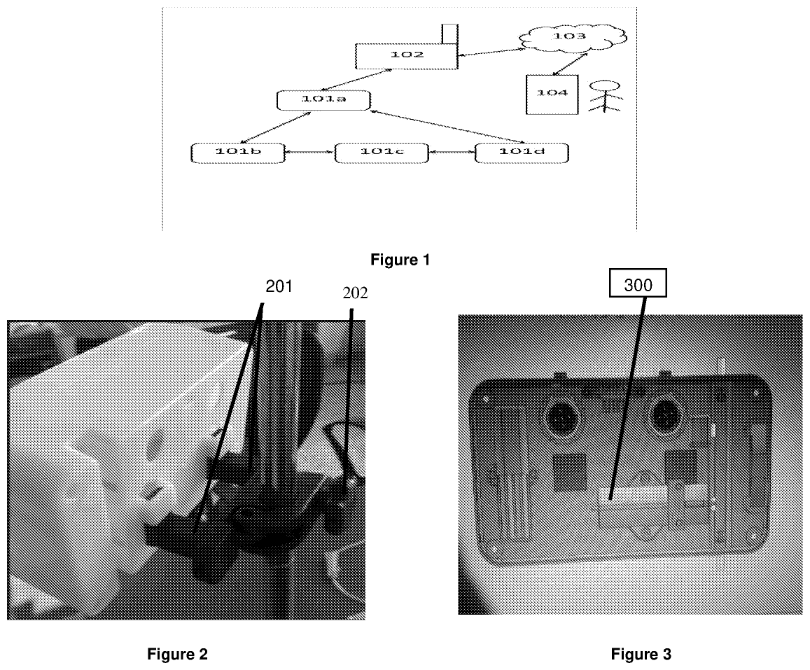

FIG. 1 shows infusion unit operating through a two way communication method that further comprises of communicating through a mesh network(105) for unit(101a, 101b, 101c, 101d) to unit(101a, 101b, 101c, 101d) communication, receiving information on web gateway(l02) with the help of Wi-Fi or GSM and transmitting the same to web server(103) and conveying the integrated information about the condition of patient on web application of suitable user friendly electronic device(104).

Referring to FIGS. 1, 2 and 3 which discloses the system comprises of a mounting clamp 201 fitted on a stand and a multifunctional intravenous drip infusion monitoring and controlling apparatus 101, wherein back portion of said apparatus is mounted firmly on the clamp hooks and front portion is towards the user, wherein height of said mounting clamp is adjustable according to the requirement, wherein the height is adjusted with the help of rotatable knob 202 by the process of tightening and loosening, wherein apparatus 101(a, b, c & d) is further comprises of a power source configured to operate horizontal screw tool 300, wherein said tool is further connected to microcontroller and drop rate sensors, wherein said microcontroller is capable of sending all running information of the tool to the web cloud server 103 through which one can access the intravenous fluid dripping conditions, monitor and control the same from a remote location in real time using a computer system or an electronic device 104 on a web application, wherein said electronic device is preferably a mobile phone, wherein said drop rate sensors are connected to the tool 300 capable of sensing the rate at which fluid is injected and if any abnormalities occurs, wherein motherboard of said microcontroller counts drops falling in drop chamber and slowly opens up the locked saline tube sufficient enough so that required drop rate is achieved, wherein all the information related to power source, conditions and functioning of horizontal screw tool 300 is updated on the interface of digital screen of the apparatus 101 in real time for user's convenience using computer system or an electronic device 104.

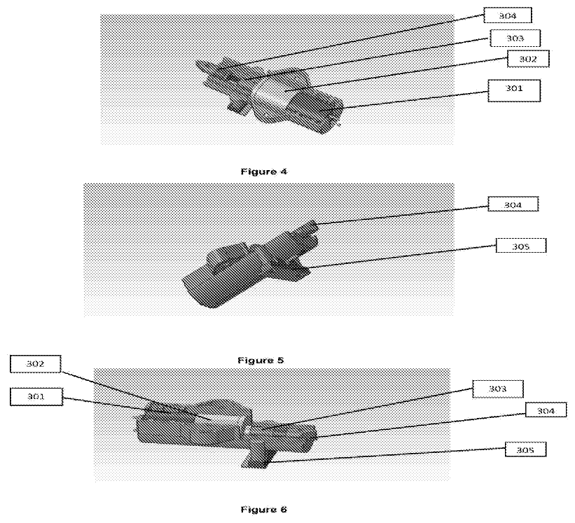

Referring to FIGS. 4, 5 and 6 that discloses a perspective views of the horizontal screw tool 300 which is capable of controlling the intravenous fluid drip accurately and efficiently by a particular mechanism, wherein said mechanism is performed by an assembly fitted in the central horizontal section of the tool further comprises of a compact DC motor 301 connected to power source, a gear box 302 in extension of said motor 301, a shaft 303 extended through said gear box 302 and, a bewild plunger 304 fitted at the end of shaft 303 by tightening it in the complementary threading of the shaft in respect of internal threading of said plunger 304, wherein said shaft 303 is threaded up to a distance equivalent to the depth of the plunger 304 provided for shaft to get fixed, wherein once the power is supplied from power source to the motor, the movement of motor 301 starts and is capable of providing more than 400 steps in 1 rpm and also provide torque of around 2 kg to enable locking the airdrop to make the drip mechanism more accurately and efficiently, wherein said assembly is placed inside the tool and said tool is mounted on a supporting base 305, wherein said base 305 is configured to be mounted at an optimum position inside the system.

The embodiments in the specification are described in a progressive manner and focus of description in each embodiment is the difference from other embodiments. For same or similar parts of each embodiment, reference may be made to each other. Because the method disclosed in the embodiment is corresponding to the unit/device/apparatus disclosed in the embodiment, the description of the method is simple. For related parts, reference may be made to the description of the apparatus.

It will be appreciated by those skilled in the art that the foregoing description was in respect of preferred embodiments and that various alterations and modifications are possible within the broad scope of the appended claims without departing from the spirit of the invention with the necessary modifications.

Based on the description of disclosed embodiments, persons skilled in the art can implement or apply the present disclosure. Various modifications of the embodiments are apparent to persons skilled in the art, and general principles defined in the specification can be implemented in other embodiments without departing from the spirit or scope of the present disclosure. Therefore, the present disclosure is not limited to the embodiments in the specification but intends to cover the most extensive scope consistent with the principle and the novel features disclosed in the specification.

1. A real time multipurpose infusion monitoring and controlling unit comprising:
Power source;
Monitor;
Micro Controller; and
Drop rate sensors,
wherein said unit is performing employing horizontal screw mechanism adapted to monitor and control the drip by locking the airdrop in the saline tube to make the drip mechanism more accurately and efficiently.

2. The infusion unit as claimed in claim 1, wherein the screw type mechanism employed for pushing and controlling the airdrop in a saline tube, wherein said mechanism is performed by an assembly comprising:
a compact DC motor;
a gear box;
a shaft and,
a plunger on the shaft,
wherein once we supply the power to the motor, the movement of motor starts which adapted to provide more than 400 steps in 1 rpm and also provide torque of around 2 kg to enable locking the airdrop to make the drip mechanism more accurately and efficiently.

3. The infusion unit as claimed in claim 2, wherein the screw type mechanism is having three positions as an air lock position, required drop rate position and zero position.

4. The infusion unit as claimed in claim 1, wherein said infusion unit is monitoring and controlling the gravity-fed drip infusions.

5. The infusion unit as claimed in claim 1, wherein said infusion unit safely monitors the intravenous delivery of all kinds of fluids including chemotherapy treatment.

6. The infusion unit as claimed in claim 1, wherein said infusion unit is compact and portable and is used anywhere in ICU as well as in general ward.

7. The infusion unit as claimed in claim 1, wherein said sensors are auto locking type universal drop rate sensor which operates with all kinds of giving sets without the use of special adaptors or other extra hardware.

8. The infusion unit as claimed in claim 1, wherein said unit is used with multiple ad-on equipments including B P machine, temperature sensing machine, ECG, Pulse check tool and the like.

9. The infusion unit as claimed in claim 1, wherein said unit has in-built embedded high end microcontroller capable of communicating unit to unit by RF data stream.

10. The infusion unit as claimed in claim 1, wherein said unit is having universal mounting means that equipment can be standardized across the hospital or institution with the unit easily switched from one treatment to another.

11. The infusion unit as claimed in claim 1, wherein said unit is communicated through web data logging, SMS, Mesh network, web gate way, web server, web application and other smart applications.

12. The infusion unit as claimed in claim 1, wherein said unit works on Battery, main, or solar or other renewal energy power.
